(19) United States
(12) Patent Application Publication
(10) Pub. No.: US 2025/0090674 A1
(43) Pub. Date: Mar. 20, 2025

(54) CARRIER MATERIAL FOR NONTOXIC POLYCATIONIC POLYMER

(71) Applicant: ZHEJIANG UNIVERSITY, Hangzhou (CN)

(72) Inventors: Changyou GAO, Hangzhou (CN); Qiaoxuan WANG, Hangzhou (CN)

(21) Appl. No.: 18/963,732

(22) Filed: Nov. 28, 2024

Related U.S. Application Data
(63) Continuation of application No. PCT/CN2022/097160, filed on Jun. 6, 2022.

(30) Foreign Application Priority Data
May 30, 2022 (CN) 202210598878.4

(51) Int. Cl. A61K 47/64; C12N 15/88

(52) U.S. Cl. CPC A61K 47/645; C12N 15/88

(57) ABSTRACT

A carrier material for a nontoxic polycationic polymer provided. The polycationic polymer carrier material is water-soluble hyperbranched polylysine with a molecular weight of 5000-6000 g/mol, and can be used as a delivery carrier for various functional molecules. The polycationic polymer is able to load and deliver small molecule drugs, macromolecular functional proteins and nucleic acid molecules such as DNA and mRNA, so as to effectively play the functions of the loaded molecules in a target area, but the carrier itself is not toxic within the use concentration range.

### CROSS-REFERENCE TO RELATED APPLICATIONS

The present application is a National Stage of International Application No. PCT/CN2022/097160 filed Jun. 6, 2022, which claims a priority to Chinese Patent Application No. 2022105988784, filed on May 30, 2022, both of which are hereby incorporated by reference in their entireties.

### TECHNICAL FIELD

The present disclosure relates to a new use of a carrier material for a nontoxic polycationic polymer, which belongs to the technical field of polymers.

### BACKGROUND

Drug Delivery Systems (DDSs) have shown unique functions in recent years and can meet the needs of different diseases. A drug delivery system refers to a pharmaceutical preparation that can improve the therapeutic effect of drugs through different forms of administration, which can reduce the toxic and side effects of drugs and improve the bioavailability of drugs. Traditional pharmaceutical preparations include capsules, injections, tablets, and the like. Some new preparation materials such as liposomes, micelles, gels, and microcapsules have been the focus of research in recent years.

Biological macromolecules such as hyaluronic acid, chitosan, and polyamino acid, as natural active components in vivo, not only have good biocompatibility, but also have the advantages of easy degradation, long plasma half-life, low immunogenicity, and high targeting, and are ideal drug carriers in current research. Among them, polyamino acids such as poly-arginine, polylysine, and poly-glutamic acid are synthetic peptides with a protein-like structure with amino acids (arginine, lysine, glutamic acid, etc.) as structural units, which have good biocompatibility and can be biodegradable in vivo, and neither the polymer itself nor the degradation products are toxic.

Hyperbranched polylysine (HBPL) is a kind of highly branched three-dimensional biomacromolecule synthesized from lysine, which can be synthesized conveniently and has many branching points, and the molecular chain is not easily entangled, and the viscosity does not change with the increase of molecular weight. HBPL is rich in amino functional groups, and it is rich in positive charges under human conditions and thus can be modified easily, which is beneficial to the synthesis of various functional materials. As a carrier material, it is non-cytotoxic, easy to degrade, and its degradation products are essential amino acid nutrients for the human body. It has simple operation and wide application fields, and can overcome the problems of slow degradation rate, complex manufacturing process, and considerable safety in vivo application of traditional carriers.

### SUMMARY

An object of this disclosure is to provide a new use of a polycationic polymer carrier. The polymer also has the ability to both load and deliver small molecule drugs, functional proteins or nucleic acid molecules such as DNA and mRNA.

The technical solution provided by the present disclosure is as follows:

A carrier material for a nontoxic polycationic polymer, wherein the polycationic polymer is water-soluble hyperbranched polylysine with a molecular weight of 5000-6000 g/mol;

Because the hyperbranched polylysine in the carrier material according to the present disclosure is rich in amino groups, it can react with many small molecules or polymers with active functional groups such as carbon-carbon double bonds, aldehyde groups, carboxyl groups and the like under different conditions to prepare different forms of drug delivery carriers (hydrogels, nanoparticles, polyelectrolyte complexes and the like).

The carrier material of the present disclosure can compound functional drugs, proteins and nucleic acids through electrostatic, hydrogen bonding or hydrophobic interactions and allow them to play their corresponding roles.

The drugs include, but are not limited to, broad-spectrum antibiotics minocycline hydrochloride and doxycycline.

The functional proteins include, but are not limited to, bovine serum albumin, inflammatory factor adsorption protein, matrix metalloproteinase response polypeptide (MMP) and H7N9 avian influenza vaccine.

The nucleic acid types include, but are not limited to, plasmid DNA and mRNA.

The present disclosure has the beneficial effects that:
(1) hyperbranched polylysine can be prepared into different forms of materials such as hydrogels, nanoparticles or polyelectrolyte complexes by covalent or non-covalent means;
(2) the carrier material containing hyperbranched polylysine can compound and sustainly release small molecular drugs;
(3) the carrier material containing hyperbranched polylysine can be combined with functional protein through an electrostatic interaction; and
(4) the carrier material containing hyperbranched polylysine can be used as a gene carrier to transfect cells with DNA and mRNA, and has good gene transfection efficiency.

### BRIEF DESCRIPTION OF DRAWINGS

FIG. 1 is a schematic structural diagram of hyperbranched polylysine;

FIG. 2 is a drug release curve of a hyperbranched polylysine hydrogel loaded with minocycline;

FIG. 3 is a HBPL hydrogel carrier material prepared in Example 1 of the present disclosure;

FIG. 4 is an electron microscope schematic diagram of hyperbranched polylysine micro-nano particles loaded with bovine serum albumin;

FIG. 5 shows the particle size of hyperbranched polylysine polyelectrolyte complex loaded with DNA/mRNA;

FIG. 6 shows fluorescence images of HeLa cells transfected with hyperbranched polylysine loaded DNA (a) or mRNA (b);

FIG. 7 shows fluorescence images of 293T cells, CHO-K1 cells, HCT116 cells and HeLa cells transfected with hyperbranched polylysine loaded DNA; scale: 100 μm.

### DETAILED DESCRIPTION OF EMBODIMENTS

The present disclosure will be further described with reference to specific examples and drawings in the specification.

According to the present disclosure, a hydrogel carrier material containing hyperbranched polylysine is prepared. The hydrogel carrier material is formed by the functional groups on a gel-forming component and cyclodextrin molecules grafted on hyperbranched polylysine through Schiff base or supramolecular action; the gel-forming component is hyaluronate aldehydel; the molecular weight of the hyperbranched polylysine is 5000-6000 g/mol; according to an example of the present disclosure, the method comprises the following steps:
(1) dissolving β-cyclodextrin and sodium hydroxide in water, and adding a chloroacetic acid solution to react to obtain carboxyl cyclodextrin;
(2) dissolving hyperbranched polylysine, 1-(3-dimethylaminopropyl)-3-ethylcarbodiimide hydrochloride, N-hydroxysuccinimide and carboxyl cyclodextrin in a phosphate buffer to react to obtain hyperbranched polylysine grafted with cyclodextrin;
(3) preparing the hyperbranched polylysine grafted with cyclodextrin into a solution A, and preparing the gel-forming component, hyaluronate aldehydel, into a solution B;
(4) mixing the solution A and the solution B in (3) to obtain the hydrogel carrier material containing hyperbranched polylysine; the material is used for loading a drug for treating brain injury, and the drug is minocycline.

### Example 1: Hyperbranched Polylysine Hydrogel Loaded with Minocycline

### (1) Preparation of Carboxymethyl Cyclodextrin (CM-β-CD)

11.34 g of β-cyclodextrin and 9.3 g of sodium hydroxide were weighed and dissolved in 37 mL of deionized water, and stirred at 50° C. for dissolution. Then, 27 mL of 16.3% chloroacetic acid was dripped into the cyclodextrin solution with a separatory funnel within 30 min and reacted for 5 h. After cooling the solution to room temperature, the mixture was allowed to stand at 4° C. overnight, the pH was adjusted to 6-7, the solution was precipitated with methanol, and filtered and washed to obtain a final product.

### (2) Preparation of Aldehyde Substituted Hyaluronic Acid (HA-ALH)

—OH on hyaluronic acid was oxidized to —CHO by sodium periodate. 1 g of HA was weighed and added in a three-neck flask, and 100 mL of deionized water was added and the mixture was stirred until HA was completely dissolved. The flask was put in a water bath at 37° C. under stirring, and 5 mL of 0.5 M sodium periodate (NaIO₄) aqueous solution was slowly dropped into the flask in 30 min to be reacted in the water bath at 37° C. for 8 h in a dark place. After the reaction was completed, 140 μL of ethylene glycol was added to stop the unreacted NaIO₄. After reacting at 37° C. for 1 h, the reaction solution was poured into 800 mL of ice ethanol for precipitation, and the precipitate was collected by centrifugation. The precipitate was subjected to redissolution and dialysis with pure water for 7 days, followed by freeze drying to obtain a final product.

### (3) Hyperbranched Polylysine and Hyaluronate Aldehydel Forming Hydrogel.

A hydrogel was obtained by the reaction of the amino group of hyperbranched polylysine with the aldehyde of hyaluronate aldehydel by a Schiff base action. The structural schematic diagram of hyperbranched polylysine is shown in FIG. 1. The hyperbranched polylysine and hyaluronate aldehydel were mixed into a 10% aqueous solution, and 33 μL of a hyaluronate aldehydel aqueous solution and 66 μL of minocycline hydrochloride/hyperbranched polylysine aqueous solution were mixed and stirred, and the gel could be quickly formed within 30 s. Then, the hydrogel was put into a 5 mL centrifuge tube, 2 mL of a phosphate buffer (pH=7.2-7.4) was added, 0.4 mL of the solution was taken for ultraviolet analysis according to the self-determined time nodes, and 0.4 mL of the phosphate buffer was supplemented at the same time.

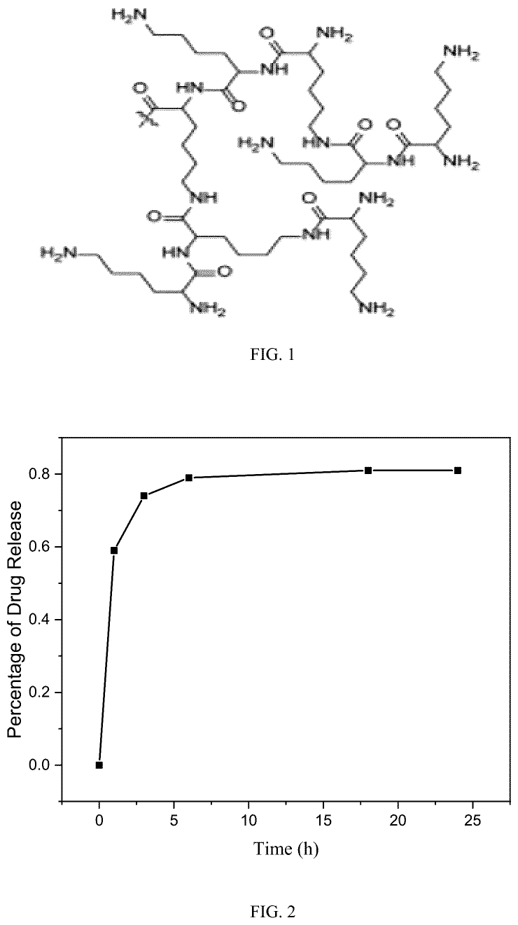

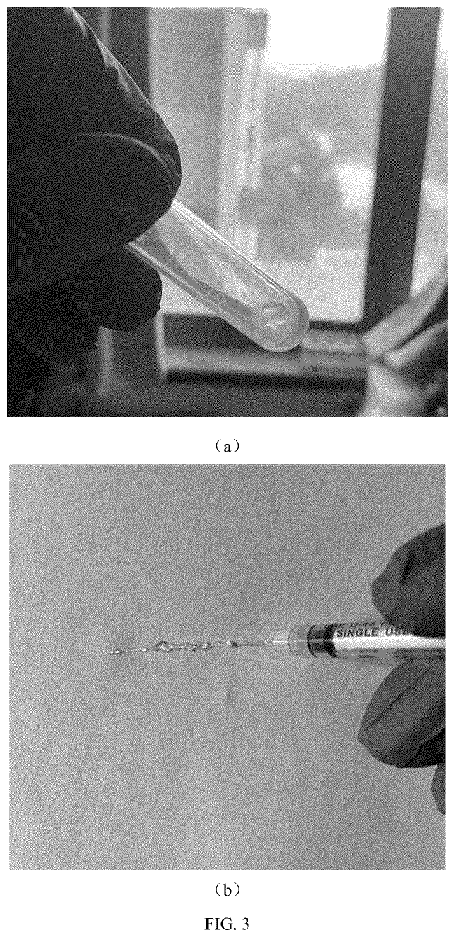

The drug release curve of minocycline is shown in FIG. 2. After being loaded by the hydrogel, the drug release of minocycline reached 80% after about 6 hours. The prepared hydrogel is shown in FIG. 3, and FIG. 3(b) shows that the hydrogel can be injected.

The HBPL hydrogel material prepared here has good biocompatibility, injectability, and no biotoxicity. The hydrogel is formed by cyclodextrin and adamantane grafted on the component through the host-guest action or by oxidizing the aldehyde group of hyaluronic acid and the amino group of HBPL through the Schiff base action. This hydrogel can be gelled by simple physical mixing, which has lower gelation conditions and simpler operation compared with medical hydrogels. At the same time, the hydrogel has suitable degradation performance, and the degradation products are nontoxic and have certain nutritional value; it can form electrostatic or hydrophobic interaction with drug proteins, which is beneficial to the sustained release of drug proteins and can maintain the activity of drug proteins. When injected into the injured site, the hydrogel can inhibit the growth of bacteria, thus reducing the problem of bacterial infection caused by surgery, promoting the survival of neuron cells, reducing the infiltration of astrocytes and microglia, reducing the nerve damage caused by excitotoxicity, and promoting neuroangiogenesis in the injured site.

### Example 2: Protein-Loaded Hyperbranched Polylysine Micro-Nano Particles

The hyperbranched polylysine was fully dissolved in ultrapure water to obtain a hyperbranched polylysine solution with a concentration of 5 mg/mL. The nonionic surfactant (SPAN 80) was dissolved in ultrapure water to obtain an emulsifier solution with a concentration of 5 mg/mL. Bovine serum albumin (BSA) was dissolved in a phosphate buffer to obtain a protein solution with a concentration of 5 mg/mL. The hyperbranched polylysine solution and emulsifier solution were mixed into a transparent mixed solution, and then the solution in which the proteins were dissolved was dripped into the mixed solution at a uniform speed, and magnetic stirring was carried out for a certain period of time to obtain a hyperbranched polylysine micro-nano carrier suspension. Centrifugation was carried out to discard the supernatant, and the precipitate was washed with deionized water to obtain the water dispersion of hyperbranched polylysine micro-nano carrier with the dispersant and free proteins removed.

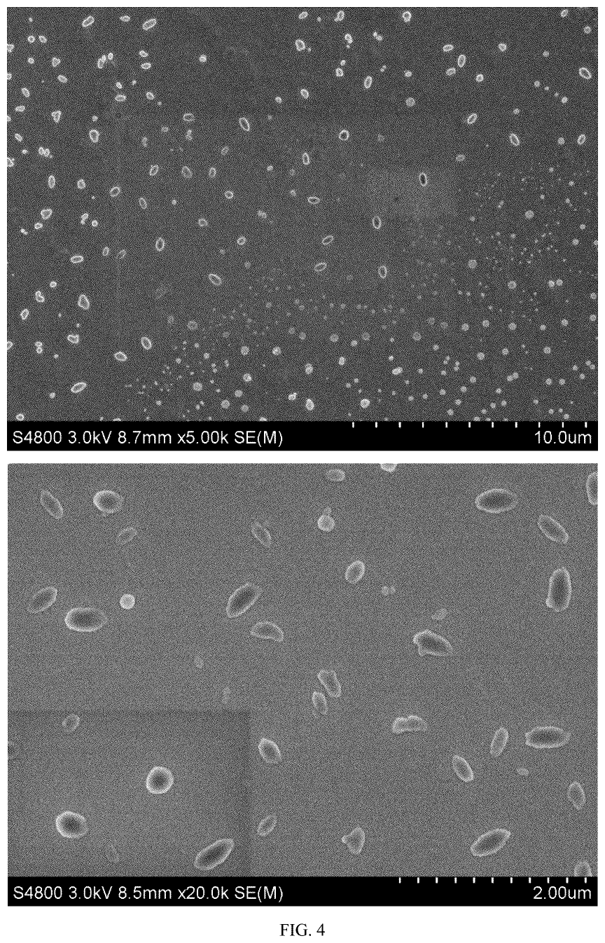

As shown in FIG. 4, the prepared micro-nano particles loaded with bovine serum albumin are shown by field emission scanning electron microscopy.

### Example 3: Hyperbranched Polylysine Complex Loaded with Nanoscale Nucleic Acid Polyelectrolytes for Cell Transfection

When the molecular weight of HBPL is too low, HBPL cannot be effectively loaded with nucleic acid, and when the molecular weight is too high, its cytotoxicity will be enhanced, therefore, it is preferable that the molecular weight of HBPL used is 5000-6000 g/moL.

The hyperbranched polylysine powder was dissolved in a phosphate buffer (PBS, pH=7.2-7.4) to prepare a PBS solution of 1 mg/mL hyperbranched polylysine, which was diluted to 10 μg/mL, 50 μg/mL, 100 μg/mL, 250 μg/mL and 500 μg/mL respectively. The diluted hyperbranched polylysine PBS solution was mixed with 100 μg/mL plasmid DNA or mRNA in equal volume, and the plasmid DNA or mRNA used contained gene fragments capable of expressing enhanced green fluorescent protein. The mixed solution was vortexed for 10 s and then allowed to stand for 30 min to prepare a HBPL/DNA or HBPL/mRNA polyelectrolyte complex with a mass ratio of HBPL to DNA or mRNA of 0.1:1-10:1.

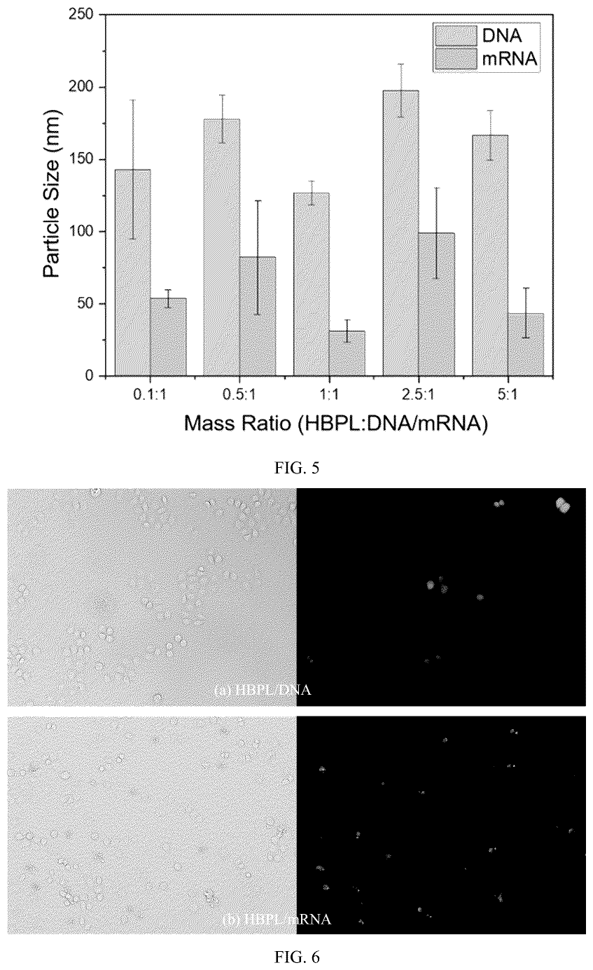

The average particle size of the prepared HBPL/DNA or HBPL/mRNA polyelectrolyte complex is shown in FIG. 5.

Hela cells were resuscitated, and when the cells occupied about 80% of the culture plate area, HeLa cells were digested and separated, and seeded on a 6-well plate according to the number of 5×10⁴/well. The cells were incubated for 24 h with a complete medium without penicillin and streptomycin, washed with PBS for 3 times, then added to a serum-free medium.

Then 200 μL of a polyelectrolyte complex solution of HBPL/DNA or HBPL/mRNA with a mass ratio of 0.1:1-5:1 was added into the 6-well plate and mixed evenly, so that the final DNA or mRNA content per well was 5 g/mL. The well plate was incubated in a 5% CO2 incubator at 37° C. for 24 hours, and the transfection was observed by fluorescence microscope.

Similarly, the prepared polyelectrolyte complex solution was added to 293T cells, CHO-K1 cells, HCT116 cells and HeLa cells by the same operations as above, and the transfection effects in different cells were compared.

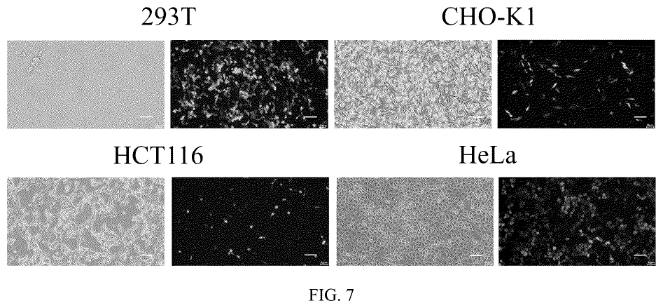

As shown in FIG. 6, it shows fluorescence microscope images of Hela cells transfected with HBPL/DNA and HBPL/mRNA (with a mass ratio of 2:1), in which green fluorescence represents eGFP expression. From the figure, it can be seen that HBPL/DNA and HBPL/mRNA can deliver DNA or mRNA into Hela cells and express DNA or mRNA.

As shown in FIG. 7, it shows fluorescence microscope images of 293T cells, CHO-K1 cells, HCT116 cells and HeLa cells transfected with HBPL/DNA (with a mass ratio of 10:1), in which green fluorescence represents eGFP expression. From the figure, it can be seen that HBPL/DNA can deliver DNA to 293T cells, CHO-K1 cells, HCT116 cells and HeLa cells and express the function of DNA.

What is claimed is:

1. A carrier material for a nontoxic polycationic polymer, wherein the polycationic polymer is water-soluble hyperbranched polylysine with a molecular weight of 5000-6000 g/mol, which is able to serve as a molecular delivery carrier with functions of both loading and delivering a small molecular drug, a macromolecular functional protein and a nucleic acid molecule, and has no cytotoxicity.

2. The carrier material for a nontoxic polycationic polymer according to claim 1, wherein the polycationic polymer compounds a functional substance such as drugs through an electrostatic action or a hydrophobic action, to realize delivery of the functional substance, or the functional substance is directly physically encapsulated in a hyperbranched network or connected on the surface of hyperbranched macromolecule to realize the delivery.

3. The carrier material for a nontoxic polycationic polymer according to claim 1, wherein the drug is selected from the group consisting of minocycline and doxycycline.

4. The carrier material for a nontoxic polycationic polymer according to claim 1, wherein the polymer, as a carrier, is compounded with a negatively charged functional protein by an electrostatic action.

5. The carrier material for a nontoxic polycationic polymer according to claim 1, wherein the functional protein comprises bovine serum albumin, inflammatory factor adsorption protein, matrix metalloproteinase response polypeptide, and H7N9 avian influenza vaccine.

6. The carrier material for a nontoxic polycationic polymer according to claim 1, wherein the polymer is able to serve as a delivery carrier for the gene or nucleic acid vaccine, which is combined with one or more of DNA and mRNA by an electrostatic action, to form a nano-polyelectrolyte complex.

7. A delivery carrier for the gene or nucleic acid vaccine according to claim 6, wherein the nano-polyelectrolyte complex is capable of penetrating a cell membrane and entering the cytoplasm or nucleus to realize expression of nucleic acid.
